SEQUENCE LISTING (4 sequences)

SEQ ID NO: 1 - DNA, 39 bp, synthetic construct
  source 1..39: mol_type = other DNA
cggtttgtttgggtttgggtttgggtttgggtttgggtt

SEQ ID NO: 2 - DNA, 39 bp, synthetic construct
  source 1..39: mol_type = other DNA
ggcttgccttacccttacccttacccttacccttaccct

SEQ ID NO: 3 - DNA, 21 bp, synthetic construct
  source 1..21: mol_type = other DNA
gttgggagttggactatggac

SEQ ID NO: 4 - DNA, 21 bp, synthetic construct
  source 1..21: mol_type = other DNA
tgaactgattggacacacaca

(19) United States
(12) Patent Application Publication
(10) Pub. No.: US 2025/0255281 A1
(43) Pub. Date: Aug. 14, 2025

(54) A METHOD FOR CONSTRUCTING A MOUSE MODEL WITH SHORT TELOMERE

(71) Applicant: Nanjing Medical University, Nanjing (CN)

(72) Inventors: Zhibin HU, Nanjing (CN); Hongbing SHEN, Nanjing (CN); Cheng WANG, Nanjing (CN); Yayun GU, Nanjing (CN); Juncheng DAI, Nanjing (CN)

(21) Appl. No.: 18/549,322

(22) PCT Filed: Dec. 16, 2022
(86) PCT No.: PCT/CN2022/139556

§ 371 Date: Nov. 8, 2024

(30) Foreign Application Priority Data
Oct. 27, 2022 (CN) 202211323532X

(51) Int. Cl. A01K 67/0275; A61D 19/04; C12N 5/073

(52) U.S. Cl. CPC A01K 67/0275; A61D 19/04; C12N 5/0604; A01K 2227/105; A01K 2267/03

(57) ABSTRACT

The present disclosure relates to a method for constructing a mouse model with short telomere. Specifically, the present disclosure obtains fertilized eggs by fertilizing the sperms and eggs of mice in vitro, and the fertilized eggs are cultured to the blastocyst stage in vitro and then transferred into the surrogate female mouse for development, thereby producing mice with short telomere. The method of the present disclosure does not require gene editing, has a short modeling cycle, and the effect is reliable and stable. By changing only the environment during embryo transfer, the telomere elongation process in the embryo is interfered, so as to successfully construct a progeny model with short telomere. There is no significant effect on the reproductive rate of female mice. Therefore, the present disclosure can provide a method for constructing a mouse model with short telomere for exploring the mechanism of telomere shortening and studying telomere-related phenotypes such as aging.

The present application claims the priority of the Chinese patent application (Application No. 202211323532X) filed on Oct. 27, 2022.

### FIELD OF THE INVENTION

The present disclosure belongs to the field of animal model construction technology, and specifically relates to a method for constructing a mouse model with short telomere.

### BACKGROUND OF THE INVENTION

Telomeres are DNA-protein complexes present at the ends of chromosomes in eukaryotic cells that act to protect the genome. As cells proliferate, telomeres continue to shorten, and when the end of chromosomes loses the protection of telomeres, the apoptosis mechanism is activated. Therefore, telomeres are attracting attention as a biomarker of aging. The association between short telomeres and tumors, cardiovascular diseases, metabolic diseases and longevity has been reported, but the exact mechanism is still not clear. Therefore, there is an urgent need to establish stable animal models with short telomere, so as to promote research on the mechanism of telomere shortening and telomere-related phenotypes such as aging.

However, existing animal models with short telomere are constructed by gene editing techniques, mainly gene knockout mice characterized by Parp^{-/-} or Atm^{-/-}, as well as Tert^{-/-} or Terc^{-/-} telomerase deficient mice. The stability of some gene knockout mouse models is still controversial. Taking Parp^{-/-} mice as an example, some studies found that the telomere length of Parp1l-deficient mice was shortened, accompanied by telomere fusion. However, telomere shortening was not observed in Parp1-deficient mice constructed in other contemporaneous studies, and only slight telomere fusion appeared, suggesting that the gene-edited mouse models may have technical instability.

In addition, it is difficult to establish mouse models based on gene knockout. Besides the requirement of mature gene editing technology, the construction of such mouse models requires several generations of breeding to obtain a stable genotype, which prolongs the model establishment cycle of the mouse models.

Therefore, the construction of a mouse model with short telomere, which has stable effect, short model establishment cycle, simple model establishment method and causes no changes to the mouse genome other than telomeres, will certainly be a powerful impetus for exploring the mechanism of telomere shortening and studying telomere-related phenotypes such as aging, and it also provides a suitable animal model for interventional studies on telomere shortening.

### SUMMARY OF THE INVENTION

In view of the above technical problems, the present disclosure provides an effective, reliable, simple and easy method for constructing a mouse model with short telomere. This method does not rely on gene editing technology, but constructs a mouse model with short telomere by interfering with the naturally-occurring telomere elongation process.

Telomere length usually reduces with age, but under the action of mechanisms such as homologous recombination and telomerase, the embryo undergoes a biological process of telomere elongation during early development. The present disclosure has found that when the embryo is cultured in vitro to the blastocyst stage, the telomere length of progeny mice is significantly shortened. That is, the length of telomeres is closely related to the embryonic development environment. Based on this, the present study attempts to culture mouse embryos in vitro to the blastocyst stage and then transfer them into female mice, interfering with the telomere elongation process of embryos at early stages, so as to construct a mouse model with short telomere.

The object of the present disclosure is achieved by the following technical solutions:

A method for constructing a mouse model with short telomere, comprising: contacting the sperms with eggs of mice in vitro to obtain fertilized eggs, and culturing the fertilized eggs to the blastocyst stage in vitro; transferring the fertilized eggs into the surrogate female mouse for development, thereby delivering mice with short telomere.

As preferred by the present disclosure, the mouse is an SPF-grade mouse of various strains (e.g., a mouse of ICR strain).

As preferred by the present disclosure, prior to fertilization, the sperms are added into a conventional commercial sperm capacitation solution (e.g., TYH sperm capacitation solution, manufacturer: Aibei Biotechnology, catalog number: M2050), under the culture conditions of 37±1° C. of temperature with 4%-7% carbon dioxide content, preferably 37° C. of temperature with 5% carbon dioxide content, for 1 hour for sperm capacitation.

As preferred by the present disclosure, prior to fertilization, the cumulus-oocyte complexes (COCs) are introduced into conventional commercial fertilization solution droplets (e.g., HTF fertilization solution, manufacturer: Aibei Biotechnology, catalog number: M1150) and placed in an incubator under the culture conditions of 37±1° C. of temperature with 4%-7% carbon dioxide content, preferably 37° C. of temperature with 5% carbon dioxide content.

As preferred by the present disclosure, the in vitro fertilization method includes intracytoplasmic sperm injection (ICSI)/in vitro fertilization (IVF).

As further preferred by the present disclosure, 1-3 μl of sperm suspension is drawn from the outer edge of the sperm capacitation solution droplets into the fertilization droplets containing the COCs, and the in vitro fertilization dish is placed into an incubator for culture.

As further preferred by the present disclosure, the in vitro culture conditions include a conventional commercial cleavage-stage embryo culture medium (e.g., cleavage medium, manufacturer: Cook, catalog number: K-SICM-20, suitable for fertilized egg stage to embryos at eight-cell stage), conventional commercial blastocyst medium (e.g., blastocyst medium, manufacturer: Cook, catalog number: K-SIBM-20, suitable for embryos at eight-cell stage to blastocyst stage), under culture conditions of 37±1° C. of temperature with 4%-7% carbon dioxide content, preferably 37° C. of temperature with 5% carbon dioxide content.

As further preferred by the present disclosure, the “culturing in vitro . . . to the blastocyst stage” is performed in vitro for about 3 to 4 days.

Use of in vitro fertilization in the preparation of the mouse model with short telomere is characterized by culturing the fertilized eggs to the blastocyst stage in vitro and then transferring them into the surrogate female mouse for development, so the mice produced are mice with short telomere.

As an embodiment of the present disclosure, provided is a method for constructing a mouse model with short telomere, comprising the following steps:

(1) Sperm preparation: Paste-like sperms are picked into another sperm capacitation solution droplet, and the sperm capacitation dish is placed into an incubator for culture for 1 hour to capacitate the sperm.

(2) Egg preparation: Cumulus-oocyte complexes (COCs) are released in mineral oil. COCs are introduced into 100 μl of fertilization solution droplets using ophthalmic forceps, and placed into an incubator under culture conditions of 37° C. of temperature with 5% carbon dioxide content, until the sperm capacitation process is finished.

(3) In vitro fertilization: 1-3 μl of sperm suspension is drawn from the outer edge of the sperm capacitation solution droplets and injected into the fertilization solution droplets containing COCs. The in vitro fertilization dish is placed into an incubator for in vitro fertilization for about 6 hours under culture conditions of 37° C. of temperature with 5% carbon dioxide content.

(4) Culture and transfer of embryos: Pseudocyesis surrogate female mice are prepared during in vitro fertilization. Cleavage-stage embryo culture dishes are prepared and 100 μl of conventional commercial cleavage-stage embryo culture medium (e.g., cleavage medium, manufacturer: Cook, catalog number: K-SICM-20) is covered with mineral oil and equilibrated in an incubator for 6 hours in advance. After 6 hours of in vitro fertilization, the fertilized eggs are washed three times with in vitro fertilization solution. After washing, the eggs are observed for the male pronucleus and female pronucleus, and unfertilized eggs are removed. The fertilized eggs are transferred into the cleavage-stage embryo culture medium and placed in the cleavage-stage embryo culture dish into an incubator for culture under culture conditions of 37° C. of temperature with 5% carbon dioxide content. After about 24 hours of culture, the embryos are observed for the status, and embryos with developmental arrest are removed. Embryos that normally develop to the 2-cell stage are kept and cultured for about another 24 hours. Blastocyst culture dishes are prepared and 100 μl of conventional commercial blastocyst culture medium (e.g., blastocyst culture medium, manufacturer: Cook, catalog number: K-SIBM-20) is covered with mineral oil and equilibrated in an incubator for 6 hours in advance. The embryos are observed for developmental status, and embryos with developmental arrest are removed. Embryos that normally develop to the 8-cell stage are selected, transferred into the blastocyst culture medium and placed into an incubator. After about 24 hours of culture, the embryos are observed for developmental status, and embryos with developmental arrest are removed. Embryos that normally develop to the blastocyst stage are selected and transferred into the uterus of surrogate female mice at 15 blastocysts per female mouse. The surrogate female mice are single-housed until delivery, and mice with short telomere are obtained.

The present disclosure does not claim the methods of obtaining sperms and eggs from mice. The present disclosure claims the method for producing mice with short telomere by contacting collected sperms and eggs in vitro to obtain fertilized eggs which develop to blastocysts, and blastocysts are transferred into the surrogate female mouse for development.

After delivery from the surrogate female mouse, the mice on the first day since birth are detected for telomere length in the peripheral blood, heart, liver, brain, lung, kidney and intestinal tissues, and the results show that the telomere length in each tissue of mice is significantly shortened. The tail vein blood is collected from the mice six months since birth for detection of telomere length, and the results show the same significant shortening. A mouse model with short telomere can be successfully constructed according to the construction method disclosed in the present disclosure.

### DESCRIPTION OF THE DRAWINGS

FIG. 1 is a schematic diagram showing the construction protocol of mice with short telomere provided in the present disclosure.

FIG. 2 is a schematic diagram showing the comparison of telomere length in the peripheral blood of mice with short telomere with that in control mice on the 1st day since birth provided in the examples of the present disclosure.

FIG. 3 is a schematic diagram showing the comparison of telomere length in the brain tissue of mice with short telomere with that in control mice on the 1st day since birth provided in the examples of the present disclosure.

FIG. 4 is a schematic diagram showing the comparison of telomere length in the heart tissue of mice with short telomere with that in control mice on the 1st day since birth provided in the examples of the present disclosure.

FIG. 5 is a schematic diagram showing the comparison of telomere length in the liver tissue of mice with short telomere with that in control mice on the 1st day since birth provided in the examples of the present disclosure.

FIG. 6 is a schematic diagram showing the comparison of telomere length in the kidney tissue of mice with short telomere with that in control mice on the 1st day since birth provided in the examples of the present disclosure.

FIG. 7 is a schematic diagram showing the comparison of telomere length in the intestine tissue of mice with short telomere with that in control mice on the 1st day since birth provided in the examples of the present disclosure.

FIG. 8 is a schematic diagram showing the comparison of telomere length in the lung tissue of mice with short telomere with that in control mice on the 1st day since birth provided in the examples of the present disclosure.

FIG. 9 is a schematic diagram showing the comparison of telomere length in the peripheral blood of mice with short telomere with that in control mice 6 months since birth provided in the examples of the present disclosure.

FIG. 10 is a schematic diagram showing the comparison of telomere length in the peripheral blood of mice with short telomere (produced by using alternative culture medium) with that in control mice on the 1st day since birth provided in the examples of the present disclosure.

FIG. 11 is a schematic diagram showing the comparison of telomere length in the peripheral blood of mice with short telomere (a technical duplicate) with that in control mice on the 1st day since birth provided in the examples of the present disclosure.

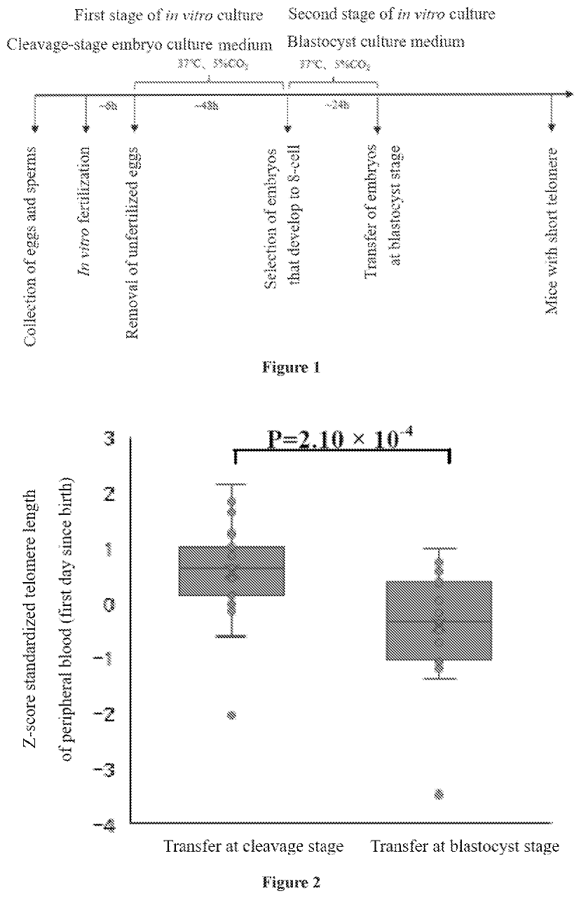

### DETAILED DESCRIPTION OF THE INVENTION

### Method for Constructing the Mouse Model

Animal models refer to non-human animals with simulated human disease manifestations established in various medicines, sciences and research.

According to some embodiments of the present disclosure, provided is a mouse model with short telomere.

Telomeres are DNA-protein complexes at the ends of chromosomes in eukaryotic cells. One of the roles of telomeres is to maintain chromosomal integrity and control the cell division cycle. Telomeres cannot be completely replicated or become lost due to multiple cell divisions, so that cells no longer divide. Therefore, severely shortened telomeres are one of the signals of cellular aging. In some infinitely replicated cells, the length of telomeres is retained after each cell division by enzymes that can synthesize telomeres.

Short telomeres refer to the fact that the telomere length in multiple tissues (including peripheral blood, liver, kidney, lung, heart, intestine, brain, etc.) in the whole body of mice born by the breeding method of the present disclosure is shorter than that of mice born by other breeding methods, and the difference is statistically significant (P-value<0.05).

The inventors have unexpectedly found that by culturing mouse embryos in vitro to a specific stage, the blastocyst stage, and then transferring them into female mice, the telomere elongation process in the early stages of the embryo is interfered with, thereby resulting in shorter telomeres in mice.

According to some embodiments of the present disclosure, provided is a method for constructing a mouse model with short telomere, comprising the following step: transferring an embryo at blastocyst stage into a surrogate female mouse to produce a mouse model with short telomere.

In the present disclosure, the blastocyst stage refers to 70 to 120 (e.g., 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100) hours after fertilization, preferably 72±2 hours after fertilization; it is required that the embryo at blastocyst stage be developed well, with intact inner cell mass and extraembryonic trophoblast, in aspect of morphology.

In the present disclosure, “after fertilization” refers to starting from the moment of obtaining the fertilized eggs (and not from the moment of contacting the sperms with the COCs)

In all embodiments, the method of the present disclosure does not involve any steps of modification of the genetic material of mouse chromosomes or mitochondria, the modification is for example, but not limited to, gene mutation, gene editing, gene knockout, mutagenesis, and introduction of exogenous nucleic acid.

In some embodiments, the mouse refers to Mus. musculus. The present disclosure has no special limitations on the strain of mice, as long as the mice are SPF grade.

SPF (Specific Pathogen Free) means that the animal does not carry a specific pathogen. Using SPF grade animals ensures that no specific disease interferes with the test results. For example, when studying the effect of a drug against aging, the animals do not carry pathogens that affect the animals' survival.

As an example, mice useful for the construction of the mouse model are any of the following strains: A/He, A/J, A/SnSf, A/WySN, AKR, AKR/A, AKR/J, AKR/N, BALB/c, B6SJLF1, B6C3F1, B6D2F1, C3H, C3He, C3Hf, C57BR, C57L, C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, C58, CBA/Br, CBA/Ca, CBA/J, CBA/st, CBA/H, CB6F1, CD2F1, CFW, DBA/1, DBA/2, FACA, FVB, ICR, KM, NIH, NIH(S), RF, SJL, SWR, TA1, TA2 and 129.

In some embodiments, provided is a method for constructing a mouse model with short telomere, comprising the following steps:

1) obtaining a sperm from a male mouse and contacting the sperm with a capacitation medium to obtain a capacitated sperm;

2) obtaining an egg from a female mouse and contacting the egg with an in vitro fertilization medium;

3) contacting the capacitated sperm obtained in step 1) with the egg obtained in step 2) in vitro to obtain a fertilized egg;

4) allowing the fertilized egg to develop to the blastocyst stage in vitro;

5) transferring the embryo at blastocyst stage into a surrogate female mouse;

6) delivering the mouse model from the surrogate female mouse; the mouse is SPF grade;

step 1) and step 2) can be interchanged in order or carried out in parallel.

In some embodiments, the sperm obtained from the male mouse can be freshly obtained, or preserved.

In some embodiments, the sperm obtained from the male mouse is freshly obtained, and the sperm is contacted with the capacitation medium.

In some embodiments, when the sperm obtained from the male mouse is cryopreserved, extracellular matrix proteins can be added to the sperm sample after thawing or quick-freezing storage. The technicians that perform IVF operation can conveniently wash the thawed sperm, concentrate the sperm by centrifugation, and then resuspend the sperm in a capacitation medium.

In some embodiments, the male mouse is 8 to 20 (e.g., 8, 9, 10, 11, 12, 13, 14, 15, 16, 17, 18, 19, 20) weeks old. In some particular embodiments, the male mouse (such as ICR) is 8 to 12 weeks old. Those skilled in the art know that as the strain varies, those skilled in the art are able to determine the equivalent week-age. In the method of the present disclosure, the inventors have found that the conservation between various strains of mice is high, and other strains of mice at the same week-age as the ICR mice can be suitably treated.

### Capacitation

Although freshly obtained sperms are morphologically mature and motile, they cannot be used for fertilization; the sperms must first undergo a maturation process called capacitation (Austin et al. The capacitation of the mammalian sperm. Nature, 170: 326 (1952); Chang et al. Fertilizing capacity of spermatozoa deposited into the fallopian tubes. Nature, 168: 697-8 (1951)).

The principle of sperm capacitation is available in prior art, for example, but not limited to, subjecting sperms to sterol efflux (Travis et al. The role of cholesterol efflux in regulating the fertilization potential of mammalian spermatozoa. The Journal of Clinical Investigation, 110:731-36 (2002)); as another example, cholesterol (and other lipids such as gangliosides) form microdomains within the plasma membrane of mouse sperms (Asano et al. Biochemical characterization of membrane fractions in murine sperm: Identification of three distinct sub-types of membrane rafts. J Cell Physiol., 218: 537-48 (2009)). As another example, in the in vitro fertilization of mice, when the concentration of GSH is 300 millimoles, the cleavage rate after fertilization is significantly improved (“Effects of glutathione on in vitro fertilization and in vitro maturation in mouse oocytes”, Master's thesis, Northeast Agricultural University, 2008).

Methods, reagents, and media for sperm capacitation are available in prior art.

In a particular embodiment, the method, reagent, and medium for capacitation are the reagent or medium in CN1893968A. As an example, the capacitation medium comprises angiotensin II amide. As an alternative, peptides containing a tripeptide motif RGD (Arg-Gly-Asp) or a tetrapeptide RGDS (Arg-Gly-Asp-Ser) can be used as the capacitation medium. RGD can be combined with angiotensin II because RGD inhibits extracellular matrix protein binding, improves the efficiency of the added angiotensin II in stimulating motility, and thus improves capacitation.

In a particular embodiment, the capacitation medium comprises: sodium salt, potassium salt, calcium salt, magnesium salt, glucose, β-cyclodextrin and polyvinyl alcohol. As a preferred example, the capacitated medium comprises: sodium chloride, potassium chloride, calcium chloride dihydrate, glucose, sodium pyruvate, magnesium sulfate heptahydrate, potassium dihydrogen phosphate, sodium bicarbonate, β-cyclodextrin, polyvinyl alcohol.

In a particular embodiment, a commercially available capacitation medium can also be used, for example, but not limited to, TYH sperm capacitation solution (Abei Biotechnology, catalog number: M2050) comprising: 119.37 mMol/L NaCl, 4.78 mMol/L KCl, 1.19 mMol/L KH₂PO₄, 1.19 mMol/L MgSO₄·7H₂O, 5.56 mMol/L glucose, 1.71 mMol/L CaCl₂·2H₂O, 25.07 mMol/L NaHCO₃, 0.5 mMol/L sodium pyruvate, 0.025 g/L gentamicin sulfate, 0.75 mMol/L methyl-β-cyclodextrin, 1 g/L polyvinyl alcohol.

In one embodiment, the sperm is contacted with the capacitation medium to obtain a capacitated sperm.

In a particular embodiment, the sperm is contacted with the capacitation medium under appropriate culture conditions for a period of time (e.g., 0.5 to 2 hours, preferably 0.5 to 1.5 hours, for example, but not limited to, 0.5, 0.6, 0.7, 0.8, 0.9, 1.0, 1.1, 1.2, 1.3, 1.4, 1.5, 1.6, 1.7, 1.8, 1.9, 2.0±10%, or the range between any two of the above values).

The appropriate culture conditions for capacitation can be those recommended by the manufacturer of the capacitation medium, or the method disclosed in CN1893968A.

As a non-limiting example, the appropriate incubation conditions for capacitation are a temperature of 30° C. to 40° C., 35° C. to 38° C., preferably 37±1° C.) with a CO₂ content of 3-10%, 4%-7%, preferably 5%.

### Collection of Cumulus-Oocyte Complexes

As used herein, the term “follicle” refers to an ovarian follicle, which is the basic unit of female reproductive biology and consists of roughly spherical aggregates of cells found in the ovaries. A follicle contains a single oocyte. Follicles periodically begin to grow and develop, and eventually ovulation occurs and usually produces a single competent oocyte.

As used herein, the term “oocyte” includes a single oocyte or an oocyte associated with one or more other cells, for example, an oocyte as part of a cumulus-oocyte complex.

As used herein, the term “cumulus cell” refers to a cell in a developing ovarian follicle that are directly close to or very close to the oocyte. Cumulus cells are involved in providing nutrients, energy and/or other requirements necessary for the oocyte to produce an available embryo at the time of fertilization.

As used herein, the term “cumulus-oocyte complex (COC)” refers to at least one oocyte and at least one cumulus cell physically bound to each other. Normally, an oocyte is surrounded by a tightly packed layer of cumulus cells, thereby forming a cumulus- oocyte complex.

In the present disclosure, the provided egg is provided in the form of a “cumulus-oocyte complex”.

In some embodiments, in order to collect preovulation COCs, prepubertal female mice are administered with chorionic gonadotropin, and then (after 30 to 50 hours, e.g., after 48 hours) dense COCs are collected from large antral follicles.

In some embodiments, the female mouse is 3 to 12 (e.g., 3, 4, 5, 6, 7, 8, 9, 10, 11, 12) weeks old. In some particular embodiments, the female mouse (such as ICR) is 4 to 5 weeks old. Those skilled in the art know that as the strain varies, those skilled in the art are able to determine the equivalent week-age.

In some embodiments, the collection and pretreatment of COCs can also employ methods in the prior art, for example the method disclosed in CN107208057A.

### IVF

IVF refers to the process of removing oocytes from female ovaries and fertilizing them with sperms in a laboratory procedure.

In some embodiments, the aforementioned capacitated sperm is contacted with the cumulus-oocyte complex in vitro to obtain a fertilized egg.

In some embodiments, under appropriate culture conditions, the capacitated sperm is contacted with the cumulus-oocyte complex in an in vitro fertilization medium for 4 to 10 hours (e.g., 4, 5, 6, 7, 8, 9, 10, or the range between any two of the aforementioned values, as an example, 5.5 to 6.5 hours) to obtain a fertilized egg. As a non-limiting example, the appropriate incubation conditions are a temperature of 30° C. to 40° C., 35° C. to 38° C., preferably 37±1° C.) with a CO₂ content of 3-10%, 4%-7%, preferably 5%.

As an example, the in vitro fertilization medium applicable to the method of the present disclosure is well known in the prior art, for example, but not limited to, the method taught in CN113817668A. The in vitro fertilization medium comprises any one selected from the group consisting of: reduced glutathione, electrolyte, carbon source, nitrogen source and a combination thereof. In an exemplary embodiment, the in vitro fertilization medium comprises any one selected from the group consisting of: sodium salt, potassium salt, magnesium salt, calcium salt, glucose, bovine serum albumin, reduced glutathione (in case of frozen sperm, preferably, the in vitro fertilization medium comprises reduced glutathione) and a combination thereof.

In a particular embodiment, a commercially available in vitro fertilization medium can also be used, for example, but not limited to, HTF fertilization solution (Abei Biotechnology, catalog number: M1150) comprising: 119.37 mMol/L NaCl, 4.78 mMol/L KCl, 1.19 mMol/L KH₂PO₄, 1.19 mMol/L MgSO₄·7H₂O, 5.56 mMol/L glucose, 1.71 mMol/L CaCl₂·2H₂O, 25.07 mMol/L NaHCO₃, 0.5 mMol/L sodium pyruvate, 0.025 g/L gentamicin sulfate, 3.98 g/sodium lactate (60% syrup), 0.0002 mmol/L phenol red.

### Obtaining Embryos at Blastocyst Stage

In some embodiments, the aforementioned obtained fertilized egg develops in vitro to the blastocyst stage.

A variety of classification criteria for mouse embryonic development are known in the art, for example, the widely used Theiler criteria.

The formation of blastocysts is the result of cleavage, and the blastomere forms a hollow spherical embryo called blastocyst. This period of the embryo is called the blastocyst stage. The blastocyst stage is usually 70 to 120 hours after fertilization (there is no significant variation among strains). Criteria for determining blastocyst stage: intact inner cell mass and extraembryonic trophoblast can be seen.

In some embodiments, under appropriate conditions, the fertilized egg is contacted with a cleavage culture medium for 40 to 54 hours (e.g., 40, 41, 42, 43, 44, 45, 46, 47, 48, 49, 50, 51, 52, 53, 54, or the range between any two of the above values; preferably 48±2 hours) to obtain an embryo at 8-cell stage. As a non-limiting example, the appropriate incubation conditions are a temperature of 30° C. to 40° C., 35° C. to 38° C., preferably 37±1° C.) with a CO₂ content of 3-10%, 4%-7%, preferably 5%. As another non-limiting example, appropriate culture conditions are those recommended by the manufacturer of the cleavage culture medium.

In some embodiments, the cleavage culture medium comprises any one selected from the group consisting of: hyaluronic acid, human serum albumin, gentamicin, bicarbonate buffer system and a combination thereof.

In some particular embodiments, commercially available cleavage culture media can also be used, for example, but not limited to, cleavage-stage embryo culture medium (Cook, catalog number: K-SICM-20).

In other particular embodiments, commercially available cleavage culture media can also be used, for example one comprising: alanine, alanyl glutamine, asparagine, aspartic acid, calcium chloride, ethylenediaminetetraacetic acid, glucose, glutamic acid, glycine, hyaluronic acid, magnesium sulfate, penicillin, potassium chloride, proline, serine, sodium bicarbonate, sodium chloride, sodium dihydrogen phosphate, sodium lactate, sodium pyruvate, taurine, water for injection; pH: 7.30±0.10, osmotic pressure: 261±5 mOsm/kg.

In some embodiments, under appropriate conditions, the embryo at 8-cell stage is contacted with a blastocyst culture medium for 20 to 28 hours (e.g., 20, 21, 22, 23, 24, 25, 26, 27, 28, or the range between any two of the above values; preferably 24±2 hours) to obtain an embryo at blastocyst stage. As a non-limiting example, the appropriate incubation conditions are a temperature of 30° C. to 40° C., 35° C. to 38° C., preferably 37+1° C.) with a CO₂ content of 3-10%, 4%-7%, preferably 5%. As another non-limiting example, appropriate culture conditions are those recommended by the manufacturer of the blastocyst culture medium.

In some embodiments, the blastocyst culture medium comprises any one selected from the group consisting of: hyaluronic acid, human serum albumin, gentamicin, bicarbonate buffer system and a combination thereof.

In some particular embodiments, commercially available blastocyst culture media can also be used, for example, but not limited to, blastocyst culture medium (Cook, catalog number: K-SIBM-20).

In some particular embodiments, commercially available blastocyst culture media can also be used, for example one comprising sodium chloride, potassium chloride, magnesium sulfate, potassium dihydrogen phosphate, magnesium chloride, sodium bicarbonate, sodium pyruvate, L-arginine·HCl, L-lysine·HCl, L-threonine, L-valine, L-leucine, L-phenylalanine, L-tryptophan, L-cystine 2HCl, L-histidine·HCl·H₂O, L-isoleucine, L-methionine, L-tyrosine, L-calcium lactate, D-glucose, alanyl glutamine, L-taurine, glycine, D-calcium pantothenate, gentamicin sulfate, human serum albumin, L-alanine, L-proline, L-serine, L-asparagine·H₂O, L-aspartic acid, L-glutamic acid, purified water.

In some embodiments, during the period from the fertilized egg to the blastocyst stage (for example, but not limited to, the 2-cell stage, the 4-cell stage, the 8-cell stage), the embryo is optionally examined to remove the abnormal ones.

### Transferring Embryos

In some embodiments, embryos at blastocyst stage are transferred into the uterus of a surrogate mouse and cultured until the production of mice with short telomere.

In some embodiments, embryos at blastocyst stage are transferred into the uterus of surrogate mice at a ratio of 15 blastocysts/mouse.

In some embodiments, the surrogate mouse is 6 to 10 weeks old, preferably 8 weeks old. In some particular embodiments, the surrogate mouse (such as ICR) is 8 weeks old. Those skilled in the art know that as the strain varies, those skilled in the art are able to determine the equivalent week-age.

Mouse Model with Short Telomere

In some embodiments, provided is a mouse model with short telomere, which is produced by the aforementioned method.

In some embodiments, the telomere length in the tissue of the mouse model with short telomere is statistically significantly shorter (or shortened, reduced, decreased) compared with the control mice.

In some embodiments, shorter (or shortened, reduced, decreased) means that telomere length is reduced by at least 10% compared with the control without applying the method of the present disclosure, and mentions may be made of, but not limited to, 10%, 20%, 30%, 40%, 50%, 60%, 70%, 80%, 90%, 100%, or the range between any two of the above values.

In other embodiments, shorter (or shortened, reduced, decreased) means that there is a statistically significant difference in the degree of reduction in telomere length compared with the control without applying the method of the present disclosure, and p is set to, for example, 0.5, 0.1, 0.05, 0.01, 0.005, 0.001, 0.0005, 0.0001, or even lower. For example, when the p-value obtained based on the measurement of two individuals (or groups) is less than a specific p-value level, it is considered that there is a statistically significant difference between the two individuals (or groups). Specifically, there is a statistically significant difference in the telomere length of the mouse model with short telomere and the control mouse at a specific p-value level.

In some embodiments, during the period of 1 day to 6 months from the date of birth of the mouse model with short telomere, the telomere length in the tissue of the mouse model with short telomere is statistically significantly shorter (or shortened, reduced, decreased) compared with the control mice.

In some embodiments, the telomere length is determined by methods well known in prior art, for example, but not limited to, terminal restriction fragment (TRF), quantitative PCR (qPCR), quantitative fluorescence in situ hybridization (Q-FISH), flow cytometry and flow fluorescence in situ hybridization (Flow FISH), single telomere length analysis (STELA), telomere length analysis based on whole genome sequencing, preferably quantitative PCR.

In some embodiments, the telomeres are from any tissue selected from the group consisting of: peripheral blood, heart, liver, brain, lung, kidney, intestine and a combination thereof.

In some embodiments, the control mouse and the mouse model are of the same strain.

In some embodiments, compared with the method for constructing the mouse model of the present disclosure, the method for producing a control mouse differs only in that the embryo is transferred into the surrogate female mouse before the blastocyst stage (e.g., the cleavage stage).

### Use

According to some embodiments, provided is the use of the mouse model with short telomere of the present disclosure in telomere research or aging research.

According to other embodiments, provided is the use of the mouse model with short telomere of the present disclosure in drug screening.

It should be understood that the numbering of the aforementioned steps is not intended to define a particular order of arrangement, and is only used to distinguish the different steps.

“Optionally” means that the feature or step that follows the term may or may not exist.

It should be understood that when a numeric range is referred to herein, for example, “A to B”, it is a concise expression, although not giving every point value within the range, it is deemed that the integers and decimals within the range have been explicitly disclosed in the text.

### EXAMPLES

### Example 1. Method for Constructing the Mouse Model with Short Telomere

### 1. Material Preparation

Sperm donor male mice (ICR strain, 8 to 12 weeks old) were single-housed one week before sperm collection. 30 minutes before sperm collection:
In the sperm capacitation dish, two microdroplets of 100 μl conventional commercial sperm capacitation solution (e.g., TYH sperm capacitation solution, manufacturer: Aibei Biotechnology, catalog number: M2050) were prepared and covered with mineral oil;
On the in vitro fertilization dish, two microdroplets of 100 μl conventional commercial in vitro fertilization solution (e.g., HTF fertilization solution, manufacturer: Aibei Biotechnology, catalog number: M1150) were prepared and covered with mineral oil. Both were balanced in an incubator at 37° C. with 5% CO₂.

### 2. Sperm Collection and Sperm Processing

8 to 12-week-old male mice were sacrificed by cervical dislocation and the abdominal cavity was cut open. The cauda epididymidis was collected and placed on sterilized filter paper, the blood, fat and other impurities were removed, and the surface of the cauda epididymidis was dried with the filter paper.

The cauda epididymidis was placed into a sperm capacitation solution microdroplet in the sperm capacitation dish, and paste-like sperms were squeezed out. The paste-like sperms were picked into another sperm capacitation solution microdroplet. The sperm capacitation dish was placed in an incubator and cultured for 1 hour to capacitate the sperms under culture conditions of 37° C. of temperature with 5% carbon dioxide content.

### 3. Egg Preparation

4 to 5-week-old female mice (the same strain) were intraperitoneally injected with pregnant mare serum gonadotropin (PMSG) at an injection amount of 5 IU/mouse for superovulation. 48 hours after PMSG injection, the mice were injected with human chorionic gonadotropin (HCG) at an injection amount of 5 IU/mouse. 15 hours after HCG injection, the female mice was sacrificed by cervical dislocation and the abdominal cavity was cut open. The fallopian tube was collected and placed into the mineral oil in the in vitro fertilization dish. The magnum tubae uterinae was incised and squeezed on both sides to completely release the cumulus-oocyte complexes (COCs) into the mineral oil. COCs were introduced into 100 μl of fertilization solution droplets using ophthalmic forceps, and placed into an incubator for the sperm capacitation process under culture conditions of 37° C. of temperature with 5% carbon dioxide content.

### 4. In Vitro Fertilization

1-3 μl of sperm suspension was drawn from the outer edge of the sperm capacitation solution droplets and injected into the fertilization solution droplets containing COCs. The in vitro fertilization dish was placed into an incubator for in vitro fertilization for about 6 hours under culture conditions of 37° C. of temperature with 5% carbon dioxide content.

### 5. Culture and Transfer of Embryos

Pseudocyesis surrogate female mice were prepared during in vitro fertilization.

Cleavage-stage embryo culture dishes were prepared and 100 μl of conventional commercial cleavage-stage embryo culture medium (e.g., cleavage medium, manufacturer: Cook, catalog number: K-SICM-20) was covered with mineral oil and equilibrated in an incubator for 6 hours in advance.

After 6 hours of in vitro fertilization, the fertilized eggs were washed three times with in vitro fertilization solution. After washing, the eggs were observed for the male pronucleus and female pronucleus, and unfertilized eggs were removed. The fertilized eggs were transferred into the cleavage-stage embryo culture medium and placed in the cleavage-stage embryo culture dish into an incubator for culture under culture conditions of 37° C. of temperature with 5% carbon dioxide content. After about 24 hours of culture, the embryos were observed for the status, and embryos with developmental arrest were removed.

Embryos that normally develop to the 2-cell stage were kept and cultured for about another 24 hours. Blastocyst culture dishes were prepared and 100 μl of conventional commercial blastocyst culture medium (e.g., blastocyst culture medium, manufacturer: Cook, catalog number: K-SIBM-20) was covered with mineral oil and equilibrated in an incubator for 6 hours in advance. The embryos were observed for developmental status, and embryos with developmental arrest were removed. Embryos that normally developed to the 8-cell stage were selected, transferred into the blastocyst culture medium and placed into an incubator.

After about 24 hours of culture, the embryos were observed for developmental status, and embryos with developmental arrest were removed. Embryos that normally developed to the blastocyst stage were selected and transferred into the uterus of surrogate female mice at 15 blastocysts per female mouse. The surrogate female mice were single-housed until delivery, and mice with short telomere were obtained.

### Example 2. Setting the Control Group

The experiment was divided into the following groups:

Group A: The method for model establishment in Example 1 was applied.

Group B: The fertilized eggs were transferred into the unilateral fallopian tube of surrogate female mice when they developed to the 2-cell stage (by the same method as above). Each female mouse was transferred with 15 embryos at 2-cell stage, and single-housed until delivery of the obtained mice.

Group C: The same method as Example 1 was applied, except that the media were replaced with those with equivalent functions, namely, cleavage culture medium (manufacturer: Vitro-Life), blastocyst culture medium (manufacturer: Vitro-Life)

Group D: The fertilized eggs were transferred into the surrogate female mice when they developed to the 2-cell stage (by the same method as Group C). Each female mouse was transferred with 15 embryos at 2-cell stage, and single-housed until delivery of the obtained mice.

### Test Example 1. Test of Telomere Length

1. On the day of delivery, some mice of Groups A and B were sacrificed by decapitation and dissected to obtain the peripheral blood, heart, liver, brain, lung, kidney and intestinal tissues. DNA was extracted and the relative telomere length (RTL) of each tissue was detected by qPCR.

2. In addition, the remaining mice of the two groups were kept and housed until six months of age. The tail vein blood was collected from both groups of mice, DNA was extracted, and RTL was detected by qPCR. The detection method was to amplify the DNA template using two separate pairs of primers:

Tel-F primer sequence:
(SEQ ID No. 1)
5′cggtttgtttgggtttgggtttgggtttgggtttgggtt3′
(300 nM),
Tel-R primer sequence:
(SEQ ID No. 2)
5′ggcttgccttacccttacccttacccttacccttaccct3′
(300 nM),

reaction conditions: 95° C., 10 min; 30 cycles: 95° C., 15 s, and then 56° C., 1 min. The individual reaction system was: 5 μl ChamQSYBR qPCR MasterMix (Manufacturer: Vazyme, catalog number: Q331-02), F and R primers (at the concentration as previously described), 20 ng DNA template, brought to 10 μl with ddH₂O.

36B4-F primer sequence:
(SEQ ID No. 3)
5′gttgggagttggactatggac3′ (300 nM),
36B4-R primer sequence:
(SEQ ID No. 4)
5′tgaactgattggacacacaca3′ (500 nM),

reaction conditions: 95° C., 10 min; 35 cycles: 95° C., 15 s, and then 52° C., 20 s, and then 72° C., 30 s. The individual reaction system was: 5 μl ChamQSYBR qPCR MasterMix, F and R primers (at the concentration as previously described), 20 ng DNA template, brought to 10 μl with ddH₂O.

3. The RTL of the samples were calculated according to the CT values of the two reactions of the sample, using the following method:

$$RTL = {2}^{- ( {CT}_{Tel} - {CT}_{36 B 4} )}$$

wherein:

RTL represents relative telomere length;

CT_{Tel} represents the number of cycles when amplification reaches a set threshold in the amplification reaction of telomeres;

CT_{36B4} represents the number of cycles when amplification reaches a set threshold in the amplification reaction of the 36B4 gene.

4. Statistical analyses were all performed by a special statistical analysis software (Rv4.0.2):

(1) Data standardization: The data were subjected to logarithmic transformation to ensure that they conform to a normal distribution. Different test batches were subjected to Z-score standardization to ensure comparability between data.

(2) The comparison of the reproductive rate of female mice between groups was performed using the chi-square test. The comparison of telomere lengths in mice between groups was performed using the Student t-test. The statistical significance level, P value, was set to 0.05, and all statistical tests were two-tailed.

5. The results showed that there was no significant difference in the reproductive rate of female mice in Group A and Group B. In the mice on the first day since birth, the telomere length of 7 different tissues, namely the peripheral blood, heart, liver, brain, lung, kidney and intestine, of mice in Group A was significantly shorter than that of Group B. After the mice reached adulthood (6 month-old), the telomere length of the peripheral blood of mice in Group A was still significantly shorter than that of Group B. The results indicated that the mouse model with short telomere was successfully constructed according to the construction method of Example 1.

### 6. Summary

The advantages of the mouse model with short telomere constructed in the present disclosure are as follows:

(1) The model establishment method for constructing a mouse model in the present disclosure is simple, the model establishment cycle is short, and there is no need for gene editing. The telomere elongation process of the embryo is affected by altering the environment during embryo transfer, so as to generate progeny with short telomere. This process has no significant effect on the reproductive rate of female mice. Therefore, the present disclosure provides an important method for constructing a mouse model for exploring the mechanism of telomere shortening and studying telomere-related phenotypes such as aging.

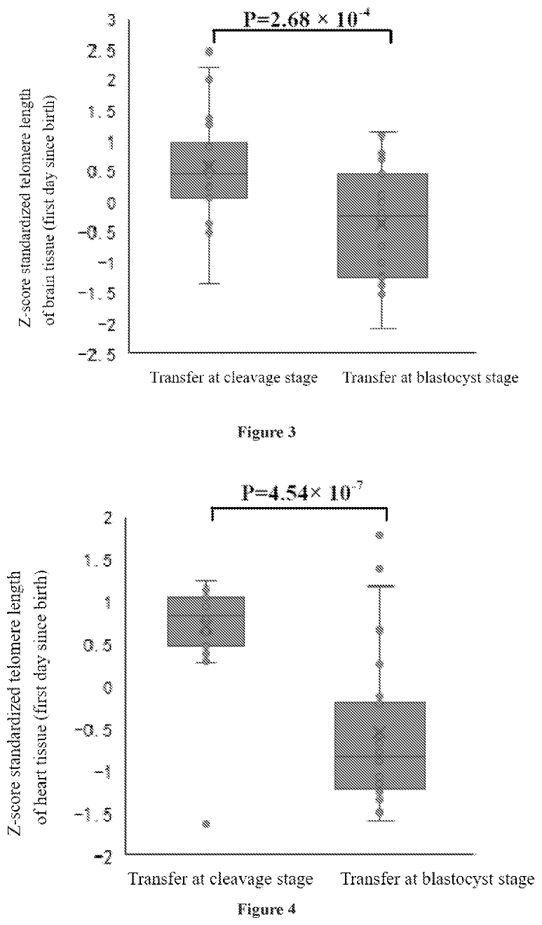

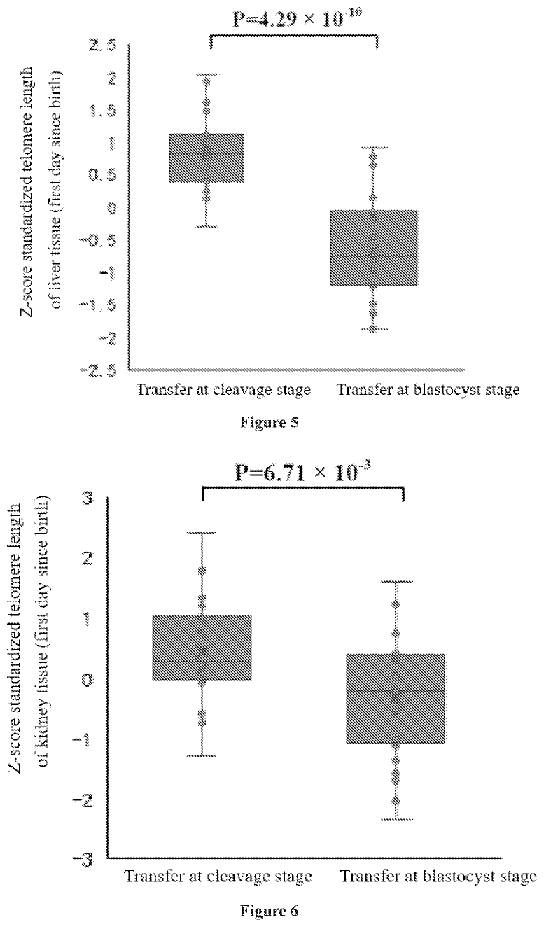

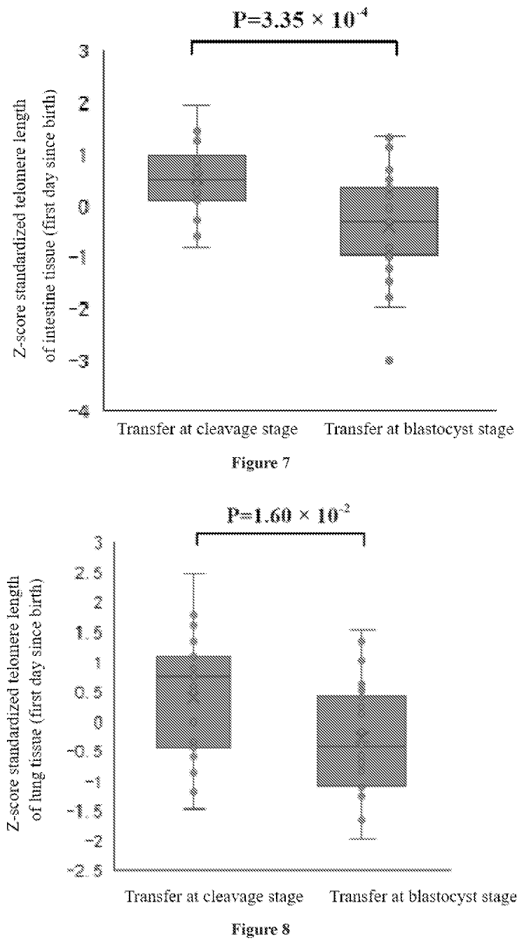

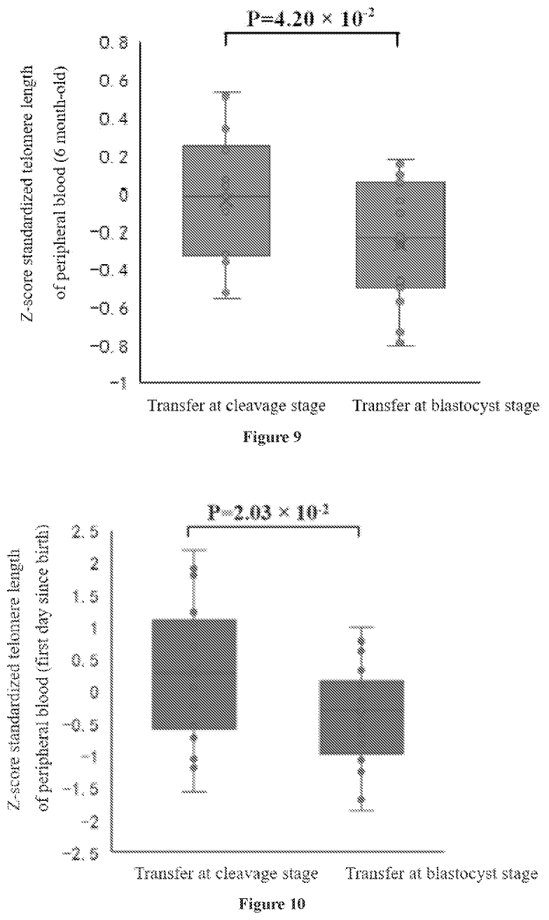

(2) The effect of model establishment of the mouse model constructed in the present disclosure is stable and reliable, and the telomere length of the peripheral blood, heart, liver, brain, lung, kidney and intestinal tissues of the constructed mouse model is significantly shortened (FIGS. 2 to 9).

(3) The mouse model with short telomere constructed based on the present disclosure can be used to simulate the embryo transfer process of assisted reproduction process in human, which is helpful to explore the mechanism of telomere shortening and to carry out intervention research on short telomeres.

### Test Example 2. Test of Telomere Length

1. On the day of delivery, the tail vein blood was collected from both groups of mice, DNA was extracted, and RTL was detected by qPCR. The detection method was the same as Test Example 1.

2. The method for RTL calculation was the same as Test Example 1.

3. The method for statistical analysis was the same as Test Example 1.

4. The results showed that in the mice on the first day since birth, the telomere length of the peripheral blood of mice in Group C was significantly shorter than that of Group D. The results indicated that the construction method according to Example 1 was not affected by the culture conditions (medium), and a mouse model with short telomere was successfully constructed (FIG. 10).

### Test Example 3. Repeatability

1. The experiment was divided into two groups:

Group E: The same method for model establishment as Example 1 was applied;

Group F: The same experimental method as Group B of Example 2 was applied.

2. Steps:
On the day of delivery, some mice of Groups E and F were sacrificed by decapitation and dissected to obtain the peripheral blood. DNA was extracted and the relative telomere length (RTL) of each tissue was detected by qPCR.
The method for RTL calculation was the same as Test Example 1.
The method for statistical analysis was the same as Test Example 1.

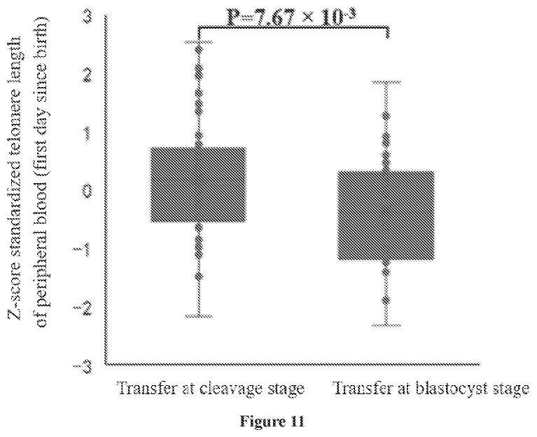

3. The results showed that in the mice on the first day since birth, the telomere length of the peripheral blood of mice in Group E was significantly shorter than that of Group F. The results indicated that the mouse model with short telomere was successfully constructed according to the construction method of Example 1 with technical stability (FIG. 11).

What is claimed is:

1. A method for constructing a mouse model with short telomere, comprising the following steps:
transferring an embryo at blastocyst stage into a surrogate female mouse to produce a mouse model with short telomere;
the blastocyst stage refers to 70 to 120 hours after fertilization, preferably 72±2 hours after fertilization;
the method does not involve modification of the genetic material of the chromosomes or mitochondria of the mouse, the modification is selected from the group consisting of: gene mutation, gene editing, gene knockout, mutagenesis, and introduction of exogenous nucleic acid.

2. The method according to claim 1, comprising the following steps:
1) obtaining a sperm from a male mouse and contacting the sperm with a capacitation medium to obtain a capacitated sperm;
2) obtaining a cumulus-oocyte complex from a female mouse;
3) contacting the capacitated sperm obtained in step 1) with the cumulus-oocyte complex in vitro to obtain a fertilized egg;
4) allowing the fertilized egg to develop to the blastocyst stage in vitro;
5) transferring the embryo at blastocyst stage into a surrogate female mouse;
6) delivering the mouse model from the surrogate female mouse;
preferably, the mouse is SPF grade;
step 1) and step 2) can be interchanged in order or carried out in parallel.

3. The method according to claim 1 or 2, wherein:
the mouse is strain selected from the group consisting of: ICR, A/He, A/J, A/SnSf, A/WySN, AKR, AKR/A, AKR/J, AKR/N, BALB/c, B6SJLF1, B6C3F1, B6D2F1, C3H, C3He, C3Hf, C57BR, C57L, C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, C58, CBA/Br, CBA/Ca, CBA/J, CBA/st, CBA/H, CB6F1, CD2F1, CFW, DBA/1, DBA/2, FACA, FVB, KM, NIH, NIH(S), RF, SJL, SWR, TA1, TA2 and 129.

4. The method according to claim 2, wherein:
the male mouse is 8 to 20 weeks old, preferably 8 to 12 weeks old;
the female mouse is an adolescent female mouse, which is 3 to 12 weeks old, preferably 4 to 5 weeks old;
the surrogate female mouse is 6 to 10 weeks old, preferably 8 weeks old.

5. The method according to claim 2, wherein in step 1):
the sperm is contacted with the capacitation medium at 35° C. to 38° C. with 4%-7% carbon dioxide content for 0.5 to 1.5 hours;
preferably, the sperm is contacted with the capacitation medium at 37° C.±1° C. with 5% carbon dioxide content for 1 hour;
preferably, the capacitation medium comprises any one selected from the group consisting of: sodium salt, potassium salt, calcium salt, magnesium salt, glucose, β-cyclodextrin, polyvinyl alcohol and a combination thereof.

6. The method according to claim 2, wherein in step 3):
the capacitated sperm is contacted with the cumulus-oocyte complex in an in vitro fertilization medium at 35° C. to 38° C. with 4%-7% carbon dioxide content for 4 to 10 hours, preferably 5.5 to 6.5 hours;
preferably, the capacitated sperm is contacted with the cumulus-oocyte complex in an in vitro fertilization medium at 37° C.±1° C. with 5% carbon dioxide content for 5.5 to 6.5 hours;
preferably, the in vitro fertilization medium comprises any one selected from the group consisting of: reduced glutathione, electrolyte, carbon source, nitrogen source and a combination thereof.

7. The method according to claim 2, wherein in step 4):
the fertilized egg is contacted with a cleavage medium at 35° C. to 38° C. with 4%-7% carbon dioxide content for 40 to 54 hours to obtain an embryo at 8-cell stage;
the embryo at 8-cell stage is contacted with a blastocyst culture medium at 35° C. to 38° C. with 4%-7% carbon dioxide content for 20 to 28 hours to obtain an embryo at blastocyst stage.

8. The method according to claim 7, wherein:
the fertilized egg is contacted with a cleavage medium at 37° C.±1° C. with 5% carbon dioxide content for 48±2 hours to obtain an embryo at 8-cell stage;
the embryo at 8-cell stage is contacted with a blastocyst culture medium at 37° C.±1° C. with 5% carbon dioxide content for 24±2 hours to obtain an embryo at blastocyst stage.

9. The method according to any one of claims 1 to 8, wherein:
the telomere length in the tissue of the mouse model is statistically significantly shorter than that of a control mice;
the tissue is selected from the group consisting of: peripheral blood, heart, liver, brain, lung, kidney, intestine and a combination thereof;
the control mouse and the mouse model are of the same strain;
preferably, the control mouse refers to a mouse produced by transferring an embryo earlier than the blastocyst stage into a surrogate female mouse; more preferably, the control mouse refers to a mouse produced by transferring an embryo at the cleavage stage into a surrogate female mouse.

10. A mouse model with short telomere obtained by the method according to any one of claims 1 to 9.

11. Use of the mouse model with short telomere according to claim 10 in telomere research or aging research.

12. Use of the mouse model with short telomere according to claim 10 in drug screening.

13. Use of a mouse embryo at blastocyst stage in the production of a mouse model with short telomere, wherein:
the blastocyst stage refers to 70 to 120 hours after fertilization, preferably 72±2 hours after fertilization;
preferably, the mouse is strain selected from the group consisting of: ICR, A/He, A/J, A/SnSf, A/WySN, AKR, AKR/A, AKR/J, AKR/N, BALB/c, B6SJLF1, B6C3F1, B6D2F1, C3H, C3He, C3Hf, C57BR, C57L, C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, C58, CBA/Br, CBA/Ca, CBA/J, CBA/st, CBA/H, CB6F1, CD2F1, CFW, DBA/1, DBA/2, FACA, FVB, KM, NIH, NIH(S), RF, SJL, SWR, TA1, TA2 and 129.
